(19) United States
(12) Patent Application Publication
(10) Pub. No.: US 2023/0408439 A1
(43) Pub. Date: Dec. 21, 2023

(54) NANOPORE SENSING SYSTEMS

(71) Applicant: ILLUMINA, INC., San Diego, CA (US)

(72) Inventors: Xu Liu, Foster City, CA (US); Lisa Savagian, San Diego, CA (US); Arvin Emadi, Hayward, CA (US)

(21) Appl. No.: 18/208,834

(22) Filed: Jun. 12, 2023

Related U.S. Application Data
(60) Provisional application No. 63/352,187, filed on Jun. 14, 2022.

(51) Int. Cl. G01N 27/327; G01N 33/487; G01N 27/419

(52) U.S. Cl. CPC G01N 27/3278; G01N 27/419; G01N 33/48721

(57) ABSTRACT

An example of a nanopore sensing system includes an application specific integrated circuit (ASIC) sensor mounted on a printed circuit board having an electrical interface with the ASIC sensor; and a nanopore sequencer formed on the ASIC sensor. The nanopore sequencer includes a redox mediator chamber having a cis electrode positioned therein; a cis well; a membrane positioned between the cis well and the redox mediator chamber, the membrane to confine a redox mediator species in the redox mediator chamber and to allow an ionic species to pass between the redox mediator chamber and the cis well; a plurality of trans wells, each including a trans electrode positioned therein; and a plurality of nanopores respectively fluidically connecting the cis well to each of the plurality of trans wells.

### CROSS REFERENCE TO RELATED APPLICATION

This application claims the benefit of U.S. Provisional Application Ser. No. 63/352,187, filed Jun. 14, 2022, the contents of which are incorporated by reference herein in their entirety.

### BACKGROUND

Various polynucleotide sequencing techniques involve performing a large number of controlled reactions on local support surfaces or within predefined reaction chambers. The designated reactions may then be observed or detected, and subsequent analysis may help identify or reveal properties of the polynucleotide involved in the reaction. Another polynucleotide sequencing technique has been developed that utilizes a nanopore, which can provide a channel for an ionic electrical current. A polynucleotide or label/tag of an incorporated nucleotide is driven into the nanopore, changing the resistivity of the nanopore. Each nucleotide (or series of nucleotides) or each label/tag (or series of labels/tags) yields a characteristic electrical signal, and the record of the signal levels corresponds to the sequence of the polynucleotide. In prior nanopore sensor devices (at t=0), the current is equally carried by the electrolyte translocating through the nanopore in opposite directions between a cis well and a trans well. However, such nanopore sequencing devices suffer from low lifetimes.

### SUMMARY

The nanopore sensing systems disclosed herein include an example of a nanopore sequencer formed on an application specific integrated circuit (ASIC) sensor. In any of the examples, the nanopore sensing system can be removably or permanently mounted on a printed circuit board having an electrical interface with the ASIC sensor.

The nanopore sensing system also utilizes polarizable and non-Faradaic electrode materials for the cis and trans electrodes and a redox mediator species for carrying sufficient current to the respective electrodes. The nanopore sensing system also includes a mechanism for confining the redox mediator species at the cis electrode so that it is physically separate from the cis well and the membrane separating the cis well from the trans wells. Physically separating the redox mediator species from the cis well keeps the redox mediator species from interfering with or otherwise deleteriously affecting the sequencing biochemistry introduced into the cis well. Physically separating the redox mediator from the membrane prevents the redox mediator species from destabilizing or otherwise deleteriously affecting the membrane.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 is a schematic illustration of one example of a nanopore sequencing system;

FIG. 2A is an exploded and perspective view of some components of another example of a nanopore sequencing system;

FIG. 2B is perspective bottom view of one of the substrates of the nanopore sequencing system of FIG. 2A, showing a first portion of a cis well;

FIG. 2C is perspective bottom view of another of the substrates of the nanopore sequencing system of FIG. 2A, showing a second portion of a cis well and a redox mediator chamber and mechanism/membrane;

FIG. 3 is a top view of another example of a nanopore sequencing system, where the cis well and the redox mediator chamber have perpendicular flow paths;

FIG. 4A is a top view of another example of a nanopore sequencing system, where the cis well and the redox mediator chamber have parallel flow paths;

FIG. 4B is a cross-sectional view taken along line 4B-4B of FIG. 4A;

FIG. 5 is a schematic illustration of two other examples of a nanopore sequencing system;

FIG. 6 is a schematic illustration of a test system used in Example 1 set forth herein;

FIG. 7 is a graph depicting an IN curve (i.e., current (pA, Y axis) versus voltage (mV, X axis)) for a single nanopore of the test system of FIG. 6;

FIG. 8 is a schematic illustration of a test cell used in Example 2 set forth herein;

FIG. 9A is a cyclic voltammetry curve (current (1 e⁻⁵ A, Y axis) versus potential (V, X axis)) one working electrode of the test cell of FIG. 8 after 10 segments;

FIG. 9B is a cyclic voltammetry curve (current (1 e⁻⁵ A, Y axis) versus potential (V, X axis)) for another working electrodes of the test cell of FIG. 8 after 10 segments;

FIG. 10 is a cyclic voltammetry curve (current (1 e⁻³ A, Y axis) versus potential (V, X axis)) for different working electrodes of the test cell of FIG. 8 after 1,000 segments;

FIG. 11A is a cyclic voltammetry curve (current (1 e⁻⁵ A, Y axis) versus potential (V, X axis)) for one working electrode of the test cell of FIG. 8 after the 1,000 segments followed by 12 hours of sitting passively and 1 additional segment;

FIG. 11B is a cyclic voltammetry curve (current (1 e⁻³ A, Y axis) versus potential (V, X axis)) for another working electrode of the test cell of FIG. 8 after the 1,000 segments followed by 12 hours of sitting passively and 1 additional segment; and

FIG. 12 is a block diagram of an example of a system for biological or chemical analysis.

### DETAILED DESCRIPTION

The technique of nanopore sequencing uses variations in electrical signal to distinguish nucleotide bases. Nanopore sensor devices include a cis well, a cis electrode, a plurality of trans wells, and a trans electrode associated with each of the plurality of trans wells. Each trans well is separated from the cis well by a membrane having a nanopore. As such, each trans well is also associated with a respective nanopore. Faradaic current between the cis electrode and trans electrode is established by redox species with or without an electrolyte buffer.

Polarizable and non-Faradaic electrode materials may be desirable for the cis and/or trans electrodes, as these materials are compatible with semiconductor foundries. These electrode materials require the use of a redox mediator species in solution to carry sufficient current at the respective electrodes. Some redox mediator species, however, can interfere with sequencing biochemistry. As one example, ferricyanide (of the ferri/ferrocyanide redox couple: Fe(CN)₆^{-3/-4}) can oxidize unsaturated amines, which are ubiquitous in proteins and enzymes (e.g., polymerases). This can interfere with polymerase activity. As other examples, redox mediator species that include metal complexes of iron (Fe), cobalt (Co), nickel (Ni), or other transition metals may interfere with protein structure and/or function. As still other examples, non-metal redox mediator species, e.g., iodine, quinone derivatives, or radical species) may deleterious affect the sequencing biochemistry and/or membrane due to the presence of radicals, the effect they have on electrolyte pH, or their ability to intercalate into the membrane.

The nanopore sensing system disclosed herein, and in particular the mechanism for confining the redox mediator species and physically separating the redox mediator species from the cis well and from the membrane, enables the use of the polarizable and non-Faradaic electrode materials and the redox mediator species without deleteriously affecting the sequencing biochemistry or the membrane. Some examples of the mechanism include ion exchange membranes, size selective membranes, and polymers of intrinsic porosity. These mechanisms are separate from the redox mediator species and can function as a physical barrier between the cis well and a redox mediator chamber that contains the cis electrode and the redox mediator species. Another example of the mechanism includes a redox active polymer coating. This mechanism is the redox mediator species and can be coated on the cis electrode.

The examples of the nanopore sensing system disclosed herein include the nanopore sequencer formed on the ASIC sensor. Thus, at least some of the sensing electronics are integrally formed with the nanopore sequencer. The ASIC sensor achieves high-throughput sensing with from 1,000 channels (trans wells and associated electronics) to 1,000,000 channels (trans wells and associated electronics).

### Definitions

It is to be understood that terms used herein will take on their ordinary meaning in the relevant art unless specified otherwise. Several terms used herein and their meanings are set forth below.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the nanopore sensor device and/or the various components of the nanopore sensor device. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range from about 50 mM to about 500 mM should be interpreted to include not only the explicitly recited limits of from about 50 mM to about 500 mM, but also to include individual values, such as about 100 mM, about 335 mM, about 400.5 mM, about 490 mM, etc., and sub-ranges, such as from about 75 mM to about 475 mM, from about 200 mM to about 300 mM, etc. Furthermore, when “about” and/or “substantially” are/is utilized to describe a value, this is meant to encompass minor variations (up to +/−10%) from the stated value.

As used herein, the terms “fluidically connecting,” “fluid communication,” “fluidically coupled,” and the like refer to two spatial regions being connected together such that a liquid or gas may flow between the two spatial regions. For example, a cis well may be fluidically connected to a trans well or a plurality of trans wells, such that at least some ions may transit between the connected wells. The two spatial regions may be in fluid communication through a nanopore, or through one or more valves, restrictors, or other fluidic components that are to control or regulate a transit of ions through a system.

As used herein, the term “interstitial region” refers to an area in a substrate or a membrane, or an area on a surface that separates other areas, regions, or features associated with the support or membrane or surface. For example, an interstitial region of a membrane can separate one nanopore of an array from another nanopore of the array. The two areas, regions, or features that are separated from each other can be discrete, i.e., lacking physical contact with each other. In many examples, the interstitial region is continuous whereas the areas, regions, or features are discrete, for example, as is the case for a plurality of nanopores defined in an otherwise continuous membrane, or for a plurality of wells defined in an otherwise continuous support. The separation provided by an interstitial region can be partial or full separation. Interstitial regions may be formed of a material or have surface chemistry that is different from the areas, regions, or features separated by the interstitial regions. For example, the surface material at the interstitial regions may be a lipid material, and a nanopore formed in the lipid material can have an amount or concentration of polypeptide that exceeds the amount or concentration of polypeptide present at the interstitial regions. In some examples, the polypeptide may not be present at the interstitial regions.

As used herein, the term “membrane” refers to a non-permeable or semi-permeable barrier or other sheet that separates two liquid/gel chambers (e.g., a cis well and a trans well) which can contain the same compositions or different compositions therein. The permeability of the membrane to any given species depends upon the nature of the membrane. In some examples, the membrane may be non-permeable to ions, to electric current, and/or to fluids. For example, a lipid membrane may be impermeable to ions (i.e., does not allow any ion transport therethrough), but may be at least partially permeable to water (e.g., water diffusivity ranges from about 40 μm/s to about 100 μm/s). For another example, a synthetic/solid state membrane, such as silicon nitride, may be impermeable to ions, electric charge, and fluids (i.e., the diffusion of all of these species is zero). Any membrane may be used in accordance with the present disclosure, so long as the membrane can include a transmembrane nanoscale opening (e.g., a nanopore) and can maintain a potential difference across the membrane. The membrane may be a monolayer or a multilayer membrane. A multilayer membrane includes two or more layers, each of which is a non-permeable or semi-permeable material.

The membrane may be formed of materials of biological or non-biological origin. A material that is of biological origin refers to a material derived from or isolated from a biological environment such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

A material that is not of biological origin may also be used as the membrane. Some of these materials are solid state materials that can form a solid state membrane, and others of these materials can form a thin liquid film or membrane. The solid state membrane can be a monolayer, such as a coating or film on a supporting substrate (i.e., a solid support), or can be a free-standing element. The solid state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, polytetrafluoroethylene (PTFE), or elastomers, such as two-component addition-cure silicone rubber), and glasses. In addition, the solid state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid state materials. A graphene-containing solid state membrane can include at least one graphene layer that is a graphene nanoribbon or graphene nanogap, which can be used as an electrical sensor to characterize the target polynucleotide. The solid state membrane can be made by any suitable method. As examples, the graphene membrane can be prepared through either chemical vapor deposition (CVD) or exfoliation from graphite. Examples of suitable thin liquid film materials that may be used include diblock copolymers, triblock copolymers, such as amphiphilic PMOXA-PDMS-PMOXA ABA triblock copolymers.

As used herein, the term “nanopore” is intended to mean a hollow structure discrete from and extending across the membrane that permits ions, electric current, and/or fluids to cross from one side of the membrane to the other side of the membrane. For example, a membrane that inhibits the passage of ions or water soluble molecules can include a nanopore structure that extends across the membrane to permit the passage (through a nanoscale opening/channel extending through the nanopore structure) of the ions or water soluble molecules from one side of the membrane to the other side of the membrane. The diameter of the nanoscale opening/channel can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid state nanopores, and biological and solid state hybrid nanopores.

As used herein, the term “diameter,” when used in reference to a nanoscale opening, is intended to mean a longest straight line inscribable in a cross-section of the nanoscale opening through a centroid of the cross-section of the nanoscale opening. It is to be understood that the nanoscale opening may or may not have a circular or substantially circular cross-section (the cross-section of the nanoscale opening being substantially parallel with the cis/trans electrodes). Further, the cross-section may be regularly or irregularly shaped.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions and/or fluids to flow therethrough from one side of the membrane to the other side of the membrane. A polypeptide nanopore can be a monomer, a homopolymer, or a heteropolymer. Structures of polypeptide nanopores include, for example, an α-helix bundle nanopore and a β-barrel nanopore. Example polypeptide nanopores include α-hemolysin, Mycobacterium smegmatis porin A (MspA), gramicidin A, maltoporin, OmpF, OmpC, PhoE, Tsx, F-pilus, etc. The protein α-hemolysin is found naturally in cell membranes, where it acts as a channel for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central channel/pore.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide nanopore” is intended to include a polynucleotide that extends across the membrane, and permits ions and/or fluids to flow from one side of the membrane to the other side of the membrane. A polynucleotide pore can include, for example, a polynucleotide origami (e.g., nanoscale folding of DNA to create the nanopore).

Also as used herein, the term “solid state nanopore” is intended to mean a nanopore whose structure portion includes materials of non-biological origin (i.e., not of biological origin). A solid-state nanopore can be formed of an inorganic or organic material. Solid state nanopores include, for example, silicon nitride nanopores, silicon dioxide (SiO₂) nanopores, and graphene nanopores.

The nanopores disclosed herein may be hybrid nanopores. A “hybrid nanopore” refers to a nanopore including materials of both biological and non-biological origins. An example of a hybrid nanopore includes a polypeptide-solid state hybrid nanopore and a polynucleotide-solid state nanopore.

As used herein, the term “nanopore sensor device” or “nanopore sequencer” refers to any of the devices disclosed herein that can be used for nanopore sequencing. In the examples disclosed herein, during nanopore sequencing, the nanopore is immersed in an electrolyte and a potential difference is applied across the membrane. In an example, the potential difference is an electric potential difference. An electrical potential difference can be imposed across the membrane via a voltage source that injects or administers current to at least one of the ions of the respective solutions contained in the cis well and one or more of the trans wells.

The application of the potential difference across the nanopores may force the translocation of a nucleic acid through the nanopore. One or more signals are generated that correspond to the translocation of the nucleotide through the nanopore. Accordingly, as a target polynucleotide, or as a mononucleotide or a probe derived from the target polynucleotide or mononucleotide, transits through the nanopore, the current across the membrane changes due to base-dependent (or probe dependent) blockage of the constriction, for example. The signal from that change in current can be measured using any of a variety of methods. Each signal is unique to the species of nucleotide(s) (or probe) in the nanopore, such that the resultant signal can be used to determine a characteristic of the polynucleotide. For example, the identity of one or more species of nucleotide(s) (or probe) that produces a characteristic signal can be determined.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, conductance, capacitance, frequency, or other changes in an electrical waveform.

The term “substrate” refers to a rigid, solid support that is insoluble in an aqueous liquid and is incapable of passing a liquid absent an aperture, port, or other like liquid conduit. In the examples disclosed herein, the substrate may have wells or chambers defined therein. Examples of suitable substrates include glass and modified or functionalized glass, polymers (including acrylics, polystyrene and copolymers of styrene and other materials, polypropylene, polyethylene, polybutylene, polyurethanes, polytetrafluoroethylene (PTFE) (such as TEFLON® from Chemours), cyclic olefins/cyclo-olefin polymers (COP) (such as ZEONOR® from Zeon), polyimides, polyamides (i.e., nylon), etc.), ceramics, silica or silica-based materials, silicon and modified silicon, or photoresist materials.

A “stimulus source” refers to an electronic device that is to provide a stimulus that causes ionic current to flow through the nanopore. In one example, the stimulus source may be a current source or a voltage source coupled to the cis and/or trans electrodes. In another example, the stimulus source may be any source creating an electric field between the cis well and the trans well.

As used herein, the terms “well”, “cavity” and “chamber” are used synonymously, and refer to a discrete feature defined in the nanopore sequencer that can contain a fluid (e.g., liquid, gel, gas). A “cis well” is a common chamber that is fluidically connected to each of a plurality of trans wells through a respective nanopore. In some examples, the cis well is in selective ionic contact with a redox mediator chamber having a cis electrode positioned therein. In other examples, the cis well houses a modified cis electrode therein. Examples of an array of the present device may have one cis well or multiple cis wells. Each “trans well” is a single chamber that contains or is partially defined by its own trans electrode, and is also fluidically connected to one cis well. Each trans well is electrically isolated from each other trans well. In some examples, each trans well is connected to a respective stimulus source, and to a respective amplifier (e.g., Axopatch 200B amplifiers) to amplify electrical signals passing through respective nanopores associated with each of the trans wells. In other examples, the trans wells are connected to a single stimulus source which individually addresses the trans wells via multiplexing. Further, it is to be understood that the cross-section of a well taken parallel to a surface of a substrate at least partially defining the well can be curved, square, polygonal, hyperbolic, conical, angular, etc.

The aspects and examples set forth herein and recited in the claims can be understood in view of the above definitions.

Nanopore Sensing Systems

Several example nanopore sensing systems are disclosed herein. Each nanopore sensing system includes an application specific integrated circuit (ASIC) sensor that is mounted on a printed circuit board having an electrical interface with the ASIC sensor, and a nanopore sequencer formed on the ASIC sensor. Different examples of the nanopore sequencer include different examples of the mechanism for confining the redox mediator species.

Each of the nanopore sensing systems disclosed herein includes a fluidic portion (the nanopores sequencer), and an application specific integrated circuit (ASIC) array of individual sensors that are individually configured to detect electrical signals passing through respective nanopores associated with respective trans wells of the fluidic portion. The entire nanopore sensing system is replaceable after being used, for example, in a sequencing operation.

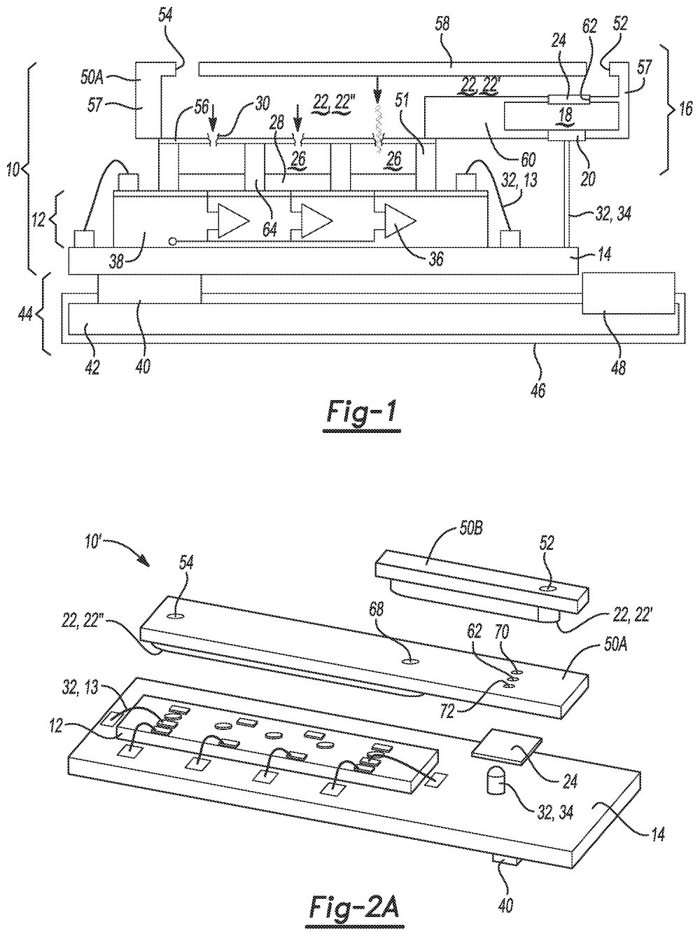

Examples of the nanopore sensing systems 10, 10′ are shown in FIG. 1 through FIG. 4B. As depicted in FIG. 1, the nanopore sensing system 10 includes the application specific integrated circuit (ASIC) sensor 12 mounted on a printed circuit board 14 having an electrical interface with the ASIC sensor 12; and a nanopore sequencer 16 formed on the ASIC sensor 12, the nanopore sequencer 16 including a redox mediator chamber 18 having a cis electrode 20 positioned therein; a cis well 22; a membrane 24 positioned between the cis well 22 and the redox mediator chamber 18, the membrane 24 to confine a redox mediator species in the redox mediator chamber 18 and to allow an ionic species to pass between the redox mediator chamber 18 and the cis well 22; a plurality of trans wells 26, each including a trans electrode 28 positioned therein; and a plurality of nanopores 30 respectively fluidically connecting the cis well 22 to each of the plurality of trans wells 26.

In an example, the ASIC sensor 12 includes an ASIC chip. The ASIC chip includes a semi-conductor wafer 38 (e.g., a silicon wafer) and electronics integrally formed within the semi-conductor wafer 38 to individually and/or collectively address each of the trans electrodes 28 of the nanopore sequencer 16. Thus, the ASIC chip may be an active silicon layer. As will be described in detail herein, each of the trans electrodes 28 is associated with a respective trans well 26 and a respective nanopore 30. Some of the electronics of the ASIC sensor 12 are schematically shown in the form of a partial circuit diagram in FIG. 1. The electronics of the ASIC sensor 12 include at least a stimulus source and a controller (not shown). The stimulus source may be coupled to each of the plurality of trans electrodes 28 either individually or via multiplexing, and the stimulus source is to cause current to flow through one or more of the nanopores 30 by addressing the trans electrode 28 associated with a respective nanopore 30. The controller is coupled to the stimulus source, and the controller is configured to individually/selectively address one of the plurality of trans electrodes 28 (using the stimulus source) to cause an ionic current to flow through the nanopore 30 connected to the addressed trans electrode 28. In one example, each of the trans electrodes 28 is electrically connected to its own set of electronics, which include the stimulus source and the controller. In another example, each of the trans electrodes 28 is electrically connected to a single stimulus source and controller, which are connected to a multiplexer. As shown in FIG. 1, the electronics may also include operational amplifier(s) 36 to amplify electrical signals passing through respective nanopores 30 associated with trans electrodes 28 that are addressed.

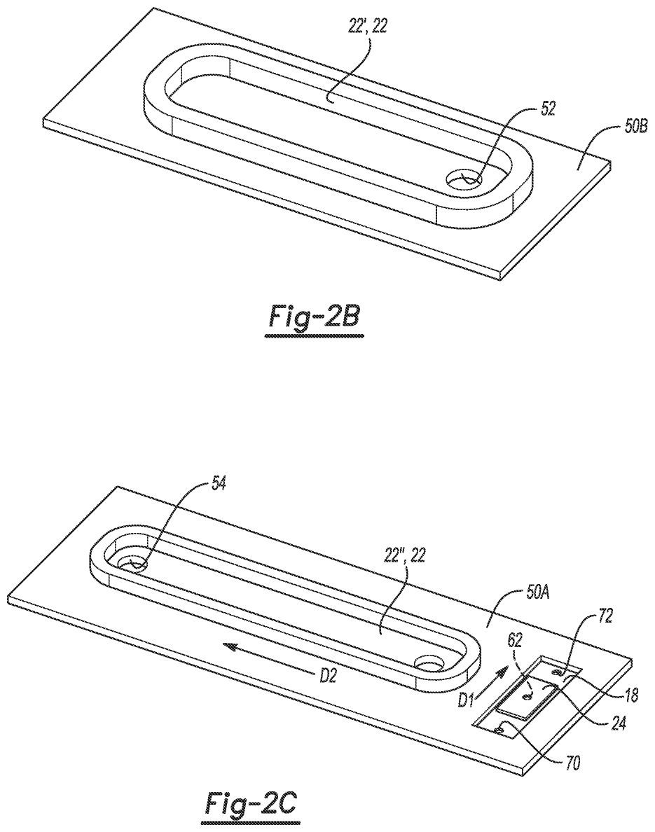

The ASIC sensor 12 is mounted on the printed circuit board 14, which has an electrical interface 32 with the ASIC sensor 12. As depicted in FIG. 1, the electrical interface 32 includes electronic circuitry that electrically connects the ASIC sensor 12 to the printed circuit board 14. It is to be understood that the electrical interface 32 may include conductive traces or pads defined on the printed circuit board 14, and/or the ASIC sensor 12. In examples, interconnections in at least a portion of a conductive path between the ASIC chip or ASIC sensor 12 and the printed circuit board 14 may be made by wire bonding as shown at reference numeral 13 in FIG. 1 and FIG. 2A. The electrical interface 32 may include wiring, ribbon cables, jumpers, connectors or any other component for electrically connecting the ASIC sensor 12 to the printed circuit board 14. The connection between the ASIC sensor 12 and the printed circuit board 14 may be disconnectable and/or reconnectable, such as provided by a multi-pin connector. At least a portion of the connection may be soldered to the printed circuit board or integrated with the ASIC sensor 12. In some examples, the electronic circuitry of the electrical interface 32 also includes components that electrically connect the cis electrode 20 of the nanopore sequencer 16 to a ground circuit connected to the printed circuit board 14.

In some examples, the ASIC sensor 12 is removably mounted on the printed circuit board 14. In this example, the electrical interface 32 includes a pogo pin connection 34 to removably connect the cis electrode 20 to a ground circuit connected to the printed circuit board 14. In an example, a clip or fastener may be used to removably mount the ASIC sensor 12 to the printed circuit board 14. In an example, the electrical interface 32 may serve as an electrical connector and a mechanical fastener to mechanically retain the ASIC sensor 12 in a position on the printed circuit board 14.

In other examples, the ASIC sensor 12 is permanently mounted on the printed circuit board 14. In an example the ASIC sensor 12 may be permanently mounted to the printed circuit board 14: using an adhesive; by soldering using through-hole joints or surface mount technology (SMT); by riveting; by staking; or by welding.

The printed circuit board 14 may include a reference electrode, which is capable of providing the current to the ASIC sensor 12, which includes a high number of trans wells 26.

The printed circuit board 14 has a connector 40 that is electrically connectable to a computing system to relay the sensor signals from each of the individual nanopores 30. Thus, the connector 40 of the printed circuit board 14 couples the entire nanopore sequencing system 10 to the computing system. In one example, the computing system is a sequencing instrument (a portion of which is shown schematically at reference numeral 44 in FIG. 1) that includes a field programmable gate array (FPGA) 42, which electrically connects via the connector 40 to the printed circuit board 14. The connector 40 may have conductors of appropriate size and materials for a predetermined number of connection cycles. In examples, the predetermined number of connection cycles may range from 1 to about 2000. In examples, the predetermined number of connection cycles may be up to about 1 million. The connector 40 may have an appropriate number of conductors to meet the data communication bandwidth/rate of the nanopore sequencing system 10. In examples, gold plated pogo pin connectors may be appropriate for up to 1 million connection cycles.

In examples, low-voltage differential signaling (LVDS) technology converts data output by the nanopore sequencing system 10 to serial data for transmission through the connector 40 to the FPGA 42. The FPGA 42 may also be connected to conductors in a USB port 48 for communicating with a computer, or other device such as a computer memory for storing data generated by the sequencing instrument 44. The computing system may include a hardware accelerator (not shown). In examples, the hardware accelerator may be local, e.g., connected via conductors to the FPGA 42. In some other examples, hardware acceleration may be accomplished in the cloud.

Referring back to the example shown in FIG. 1, the nanopore sequencer 16 includes a first substrate 50A, which functions as a lid and has the cis well 22 and the redox mediator chamber 18 defined therein; and an interposer 51 attached to the first substrate 50A and having the plurality of trans wells 26 defined therein. The first substrate 50A and the interposer 51 are separate pieces made of the same material or different materials. In these examples, the first substrate 50A and the interposer 51 are adhered together so that each trans well 26 is positioned to be in fluid communication with the common cis well 22.

The first substrate 50A may be any of the substrate materials set forth herein. The interposer 51 may be any material that is capable of being processed with micrometer accuracy for the trans wells 26. In one example, the substrate 50A may be formed of a polymer or glass and the interposer 51 may be formed of a negative photoresist (e.g., an epoxy-based negative photoresist such as SU-8). Other specific examples of the substrate 50A include ceramics and metal. Other specific examples of the interposer 51 include SUEX® (epoxy photoresist from DJ Microlaminates), polyimide, and perylene. The substrate 50A (and its features, such as the bottom portion 60 and the redox mediator chamber 18) may be fabricated using injection molding or CNC (computer numerical control) machining, and the interposer 51 (and its features, such as the trans wells 26) may be fabricated using microlithography. It is to be understood that the substrate 50A may be fabricated monolithically using, for example, additive manufacturing techniques; or the substrate 50A may be an assembly of parts joined together using appropriate joining technology, including welding, and/or adhesives. For example, a sub-assembly defining the redox mediator chamber 18 may be ultrasonically welded to the sidewall 57 after installation of the membrane 24.

Any suitable securing mechanism may be used to adhere the first substrate 50A and the interposer 51 together. Some example adhesives include a thermoset or thermoplastic polymer, tape, and a photocurable glue (e.g., acrylic based). Alternatively, the substrate 50A and the interposer 51 may be laminated together, e.g., by applying heat and/or mechanical pressure.

In other examples, the trans wells 26 may be etched into a portion of the semi-conductor wafer 38 that overlies the portion that includes the integrated electronics.

In the example shown in FIG. 1, the substrate 50A has an inlet 52 and an outlet 54 defined therein so that fluid can be introduced into the cis well 22 through the inlet 52 and can be extracted from the cis well 22 through the outlet 54. The inlet 52 and outlet 54 may be positioned at opposed ends of the cis well 22 or anywhere along the length and width of the cis well 22 that enables desirable fluid flow. The inlet 52 and outlet 54 are fluidly connected to a fluidic control system (including, e.g., reservoirs, pumps, valves, waste containers, and the like) which controls fluid introduction and expulsion.

In the example shown in FIG. 1, the cis well 22 is a fluid chamber that is defined, in part, by the sidewall 57 and a top portion 58 of the substrate 50A. This example of the cis well 22 has interior walls that are defined by the sidewall 57 and an upper surface that is defined by the top portion 58. The lower surface of the cis well 22 is defined, at least in part, by the membrane 56. It is to be understood that this portion of the lower surface of the cis well 22 has opening(s) through the nanopore(s) 30 that are positioned in the membrane 56. In the example shown in FIG. 1, the lower surface of the cis well 22 is also defined, in part, by a bottom portion 60 of the substrate 50A and by the membrane 24 positioned between the cis well 22 and the redox mediator chamber 18.

The cis well 22 may have any suitable geometry and/or dimensions. In the example shown in FIG. 1, the cis well 22 has an L-shaped geometry with a first portion 22′ that leads into the second portion 22″. The first portion 22′ is in fluid communication with the inlet 52, is adjacent to the redox mediator chamber 18, and leads into the second portion 22″. The second portion 22″ of the cis well 22 is in fluid communication with the trans wells 26. In one example, the dimensions of the first portion 22′ range from about 0.5 mm×0.1 mm×0.1 mm to about 10 mm×2 mm×2 mm, and the dimensions of the second portion 22″ range from about 1 mm×0.1 mm×0.1 mm to about 100 mm×10 mm×10 mm. In another example, the dimensions of the first portion 22′ range from about 0.5 mm×0.1 mm×0.1 mm to about 2 mm×0.5 mm×0.5 mm, and the dimensions of the second portion 22″ range from about 1 mm×0.1 mm×0.1 mm to about 30 mm×20 mm×0.8 mm.

While one cis well 22 is shown in FIG. 1, it is to be understood that the nanopore sensing system 10 may include several cis wells 22 that are fluidically isolated from one another and are fluidically connected to respective sets of trans wells 26. Multiple cis wells 22 may be desirable, for example, in order to enable the measurement of multiple samples using a single nanopore sensing system 10.

In the example shown in FIG. 1, the first portion 22′ of the cis well 22 is at least partially adjacent to the redox mediator chamber 18. In this example, the redox mediator chamber 18 is defined in a portion of the substrate 50A so that it underlies the first portion 22′ of the cis well 22. It is to be understood that the redox mediator chamber 18 and the cis well 22 are separate fluid compartments. The redox mediator chamber 18 houses the redox mediator species and the cis well 22 houses a buffer solution. During operation of the nanopore sensing system 10, the cis well 22 also receives the sequencing biochemistry.

As such, while not shown in FIG. 1, it is to be understood that this example of the substrate 50A also includes a second inlet and a second outlet defined therein so that fluid can be introduced into the redox mediator chamber 18 through the second inlet and can be extracted from the redox mediator chamber 18 through the second outlet. In this example, the second inlet and the second outlet may extend through the portion 22′ of the cis well 22, but are fluidically isolated from the portion 22′ of the cis well 22 so that the fluid (e.g., the redox mediator species) introduced into the redox mediator chamber 18 is not introduced into the cis well 22. The second inlet and second outlet may be positioned at opposed ends of the redox mediator chamber 18 or anywhere along the length and width of the redox mediator chamber 18 that enables desirable fluid flow. The second inlet and second outlet are also fluidly connected to the fluidic control system (including, e.g., reservoirs, pumps, valves, waste containers, and the like) which controls fluid introduction and expulsion.

The redox mediator chamber 18 may have any suitable geometry and/or dimensions. In the example shown in FIG. 1, the redox mediator chamber 18 has a rectangular geometry. The length of the redox mediator chamber 18 may be parallel to or perpendicular to the length of the first portion 22′ of the cis well 22. In one example, the dimensions of the redox mediator chamber 18 range from about 0.5 mm×0.5 mm×0.1 mm to about 10 mm×10 mm×2 mm. In another example, the dimensions of the redox mediator chamber 18 range from about 1 mm×1 mm×0.1 mm to about 8 mm×8 mm×2 mm.

The redox mediator chamber 18 contains or is partially defined by the cis electrode 20. In the example shown in FIG. 1, the cis electrode 20 makes up a portion of the bottom surface of the redox mediator chamber 18. The cis electrode 20 may be physically connected to portions of the substrate 50A that define the bottom surface of the redox mediator chamber 18 by an adhesive or another suitable fastening mechanism. The interface between the cis electrode 20 and the substrate 50A may seal the bottom portion of the redox mediator chamber 18. In another example, the cis electrode 20 may be positioned on the bottom portion of the redox mediator chamber 18, and the electronic component removably connecting the cis electrode 20 to the ground circuit may be integrated into the substrate 50A.

The cis electrode 20 may be any polarizable and non-Faradaic electrode material, such as gold (Au), platinum (Pt), ruthenium (Ru), carbon (C) (e.g., graphite, diamond, etc.), or rhodium (Rh).

In the example shown in FIG. 1, an aperture 62 is formed between the first portion 22′ of the cis chamber 22 and the redox mediator chamber 18. The mechanism 24 (also referred to herein as “membrane 24”) for confining the redox mediator species within the redox mediator chamber 18 is positioned in the aperture 62. In one example, the mechanism/membrane 24 is an ion exchange membrane. In another example, the mechanism/membrane 24 is a size selective membrane. In either example, the mechanism 24 may be physically connected to the portion of the substrate 50A that defines the aperture 62, for example, with an adhesive or another suitable fastening mechanism.

In one example, the mechanism/membrane 24 for confining the redox mediator species within the redox mediator chamber 18 is an ion exchange membrane. Ion exchange membranes exhibit ionic permselectivity and allow the transport of one type of ion (i.e., cation or anion). The ion exchange membrane may be anionic or cationic, depending upon the redox mediator species and cis electrode 20 that are used in the nanopore sensing system 10. In many examples, the thickness of the membrane 24 may range from about 1 μm to about 1000 μm.

Cation exchange membranes block anion movement, but allow cation movement through the mechanism/membrane 24. These types of membranes are suitable for redox mediator species that remain anionic in both the oxidized and reduced forms. Some specific examples of such redox mediator species include ferri/ferrocyanide: Fe(CN)₆^{-3/-4}, dithionate/thiosulfate: S₂O₆⁻²/S₂O₃⁻², nitrate/nitrogen dioxide: NO₃⁻/NO₂⁻, pyruvate/acetate: CH₃COCOO⁻/CH₃COO⁻. In one specific example, the mechanism/membrane 24 would block the ferri/ferrocyanide, thus maintaining the anions in the redox mediator chamber 18, while allowing the associated cationic counterions, e.g., potassium: K⁺, to carry current from the well(s) 22, 26 to the chamber 18. Any of these redox mediator species is available as a salt (e.g., potassium salt, sodium salt, etc.).

The cationic exchange membrane may be a homogeneous polycation. These polymers or co-polymers are functionalized with fixed, intrinsically anionic groups or ionizable groups that become anionic when immersed in the redox mediator species. Suitable co-polymers may include ionic repeat units that are co-polymerized with non-ionic repeat units. Examples of homogeneous cationic exchange membranes include a predominantly carbon or silicon based backbone, with pendant groups selected from the group consisting of carboxylate, sulfonate, phosphate, carbonate, bistriflimide, thiolsulfate, or precursors thereof. Any of these pendant groups may also be attached to biopolymers with cellulose or cellulose-like backbones. One specific example of a homogeneous cationic exchange membrane including a predominantly carbon based backbone is polystyrene sulfonate (PSS). Other examples of homogeneous cationic exchange membranes include perfluorosulfonic acids (PFSAs), which have a fluorinated backbone functionalized with pendant sulfonate groups. Commercially available PFSAs include NAFION™ (from The Chemours Co.) and AQUIVION™ (from Solvay). Still other examples of homogeneous cationic exchange membranes include perfuorocarboxylic acids (PFCAs), which are PFSA analogues with carboxylic acid groups instead of sulfonate groups. PFCAs may be particularly suitable for alkali metal transport (e.g., K⁺). Still other examples of a homogeneous cationic exchange membrane include n-type conjugated polymers. These polymers have a carbon-based backbone with pi conjugation that has been reduced so that negative charges are introduced into the backbone. One specific example is (Poly(benzimidazobenzophenanthroline)).

The cationic exchange membrane may alternatively be a heterogeneous membrane. These heterogeneous membranes may include an uncharged matrix (e.g., a hydrophobic polymer) impregnated or blended with an ionophore. The ionophore may be a charged anion, any of the polyanions described herein, an uncharged ionophore, or polymer particles. Some specific examples include a poly(vinyl chloride) matrix impregnated or blended with PSS, a polysulfone matrix impregnated or blended with a cation exchange resin powder, and carbon fibers impregnated or blended with sulfonated graphene. Other heterogeneous membranes include a charged matrix (e.g., a PFSA) reinforced with polypropylene or other polymeric materials, fibrous materials, ceramics (e.g., porous glass), nanomaterials, or colloidal materials. One specific example of this type of membrane is FUMASEP™ FKM (from Fuel Cell Store).

Cationic exchange membranes may be commercially available or fabricated using solution-casting, layer-by-layer assembly, in situ polymerization, hot-pressing, fiber extrusion or spinning, or surface grafting.

Anion exchange membranes block cation movement, but allow anion movement through the mechanism/membrane 24. These types of membranes are suitable for redox mediator species that remain cationic in both the oxidized and reduced forms. Some specific examples of such redox mediator species include tris(bipyridine)ruthenium(II): Ru(bpy)₃^{2+/+3+}, cobaltocene: Co(Cp)₂^{+/2+}, viologens, and trimethylaminoferrocene. The anionic exchange mechanism/membrane 24 would block the cations, thus maintaining the cations in the redox mediator chamber 18, while allowing the associated anionic counterions, e.g., chloride Cl⁻, to carry current from the chamber 18 to the well(s) 22, 26.

The anionic exchange membrane may be homogeneous or heterogeneous, depending upon the backbone components, and may also be reinforced by another material (such as those described for the cationic exchange membrane). Examples of anionic exchange membranes may include a predominantly carbon or silicon based backbone, with pendant groups selected from the group consisting of ammonium, sulfonium, phosphonium, imidazolium, or precursors thereof. Any of these pendant groups may also be attached to biopolymers with cellulose or cellulose-like backbones. Example anion exchange polymers include quaternized poly(vinylbenzyl chloride), polydiallyldimethylammonium chloride, and poly(vinylbenzyl chloride) quaternized by triphenylphosphine. One specific example of a commercially available anion exchange membrane is SUSTANION® from Dioxide Materials. Other examples of anionic exchange membranes include perfluorinated backbones that are functionalized with pendant cationic groups. One specific example of this type of membrane is FUMASEP™ FAS (from Fuel Cell Store).

In another example, the mechanism/membrane 24 for confining the redox mediator species within the redox mediator chamber 18 is a size selective membrane. In these examples, the redox mediator species is large, i.e., 1,000 Daltons or more, and the size selective membrane has pores that are too small for the redox mediator species to traverse. In this example, a membrane 24 with a molecular weight cutoff (MWCO) is at least 1,000 Daltons. In contrast, the smaller cations (the counter ion of an anionic redox mediator species) or the smaller anions (the counter ion of a cationic redox mediator species) are able to move through the pores of the mechanism/membrane 24 and into the cis well 22. In these examples, the redox mediator species may be a water-soluble redox active polycation or polyanion with a weight average molecular weight greater than the molecular weight cut off (MWCO) of the membrane, which is ideally at least 1,000 Daltons. Examples of water-soluble redox active polycations include p-type conducting polymers (e.g., poly(3,4-ethylenedioxythiophene) (PEDOT), polypyrrole, etc.), non-conjugated redox polymers, radical polymers, metallopolymers, and viologen polymers. Examples of water-soluble redox active polyanions include p-type conducting polymers with anionic functionality (e.g., carboxylayed polythiophene derivatives), n-type conducting polymers, and non-conjugated redox polymers.

Examples of suitable materials for the size exclusion membrane include dialysis tubing materials (such as SNAKESKIN™ tubing from ThermoFisher Scientific) and ultrafiltration membranes (such as AMICON® Ultra membranes available from Millipore Sigma).

The configuration of the redox mediator chamber 18 of FIG. 1 confines the redox mediator species in the chamber 18 and in contact with the cis electrode 20, while allowing the current carrying counterions to pass through to or from the cis well 22. This keeps the redox mediator species from interfering with the sequencing biochemistry that is introduced in the cis well 22.

The nanopore sensing system 10 also includes the trans wells 26, the membrane 56 separating the second portion 22′ of the cis well 22 from the trans wells 26, and respective nanopores 30 fluidly connecting each trans well 26 to the second portion 22′ of the cis well 22.

Each trans well 26 is a fluid chamber that is defined by the interposer 51. Generally, the trans wells 26 may extend through the thickness of the interposer 51 and may have openings at opposed ends of the interposer 51 (e.g., a top end where the membrane 56 is located and a bottom end where the trans electrode 28 is located). In the example shown in FIG. 1, each trans well 26 has a sidewall 64 that is defined by the interposer 51, a lower surface that is defined by the trans electrode 28, and an upper surface that is defined by the membrane 56.

The trans electrode 28, a surface of which defines the lower surface of the trans well 26, may be physically connected to the interposer 51. The trans electrode 28 may be attached to the interposer 51 (e.g., to the sidewall 64) during the formation of the trans wells 26. Microfabrication techniques that may be used to form the trans wells 26 in the interposer 51 and to form the trans electrodes 28 in the trans wells 26 include lithography, metal deposition and liftoff, dry and/or spin on film deposition, etching, etc. The interface between the trans electrode 28 and the interposer 51 may seal the lower portion of the trans well 26.

The trans electrode 28 that is used depends, at least in part, upon the redox mediator species that is to be introduced into the redox mediator chamber 18 and the transfer of electrons that is to take place at the respective electrodes 20, 28. Any of the examples of the materials for the cis electrode 20 may be used as the trans electrode 28. As specific examples, the trans electrodes 28 may be gold or platinum electrodes.

Many different layouts of the trans wells 26 may be envisaged, including regular, repeating, and non-regular patterns. In an example, the trans wells 26 are disposed in a hexagonal grid for close packing and improved density. Other layouts may include, for example, rectangular layouts, triangular layouts, and so forth. As examples, the layout or pattern can be an x-y format of trans wells 26 that are in rows and columns. In still other examples, the layout or pattern can be a random arrangement of trans wells 26.

The layout may be characterized with respect to the density of the trans wells 26 (i.e., number of trans wells 26 in a defined area of the interposer 51). For example, the trans wells 26 may be present at a density ranging from about 10 wells per mm² to about 1,000,000 wells per mm². The density may be tuned to different densities including, for example, a density of at least about 10 per mm² about 5,000 per mm², about 10,000 per mm², about 0.1 million per mm², or more. Alternatively or additionally, the density may be tuned to be no more than about 1,000,000 wells per mm², about 0.1 million per mm², about 10,000 per mm², about 5,000 per mm², or less. It is to be further understood that the density of the trans wells 26 in the interposer 51 can be between one of the lower values and one of the upper values selected from the ranges above.

The layout may also or alternatively be characterized in terms of the average pitch, i.e., the spacing from the center of a trans well 26 to the center of an adjacent trans well 26 (center-to-center spacing). The pattern can be regular such that the coefficient of variation around the average pitch is small, or the pattern can be non-regular in which case the coefficient of variation can be relatively large. In an example, the average pitch may range from about 100 nm to about 500 μm. The average pitch can be, for example, at least about 100 nm, about 5 μm, about 10 μm, about 100 μm, or more. Alternatively or additionally, the average pitch can be, for example, at most about 500 μm, about 100 μm, about 50 μm, about 10 μm, about 5 μm, or less. The average pitch for an example array including a particular pattern of trans wells 26 can be between one of the lower values and one of the upper values selected from the ranges above. In an example, the array has an average pitch (center-to-center spacing) of about 10 μm.

The trans wells 26 may be micro wells (having at least one dimension on the micron scale, e.g., about 1 μm up to, but not including, 1000 μm) or nanowells (having at least one dimension on the nanoscale, e.g., about 10 nm up to, but not including, 1000 nm). Each trans well 26 may be characterized by its aspect ratio (e.g., width or diameter divided by depth or height, respectively).

In an example, the aspect ratio of each trans well 26 may range from about 1:1 to about 1:5. In another example, the aspect ratio of each trans well 26 may range from about 1:10 to about 1:50. In an example, the aspect ratio of the trans well 26 is about 3.3.

The depth/height and width/diameter may be selected in order to obtain a desirable aspect ratio. The depth/height of each trans well 26 can be at least about 0.1 μm, about 1 μm, about 10 μm, about 100 μm, or more. Alternatively or additionally, the depth can be at most about 1,000 μm, about 100 μm, about 10 μm, about 1 μm, about 0.1 μm, or less. In one example, the depth/height ranges from about 15 μm to about 50 μm. The width/diameter of each trans well 26 can be at least about 50 nm, about 0.1 μm, about 0.5 μm, about 1 μm, about 10 μm, about 100 μm, or more. Alternatively or additionally, the width/diameter can be at most about 1,000 μm, about 100 μm, about 10 μm, about 1 μm, about 0.5 μm, about 0.1 μm, about 50 nm, or less. In one example, the width/diameter ranges from about 15 μm to about 50 μm.

Each trans well 26 has an opening (e.g., that faces the cis well 22) that is large enough to accommodate at least a portion of the membrane 56 and the nanopore 30 that is associated therewith. For example, an end of the nanopore 30 may extend through the membrane 56 and into the opening of the trans well 26.

The cis well 22, the redox mediator chamber 18, and the trans wells 26 may be fabricated using a variety of techniques, including, for example, photolithography, nanoimprint lithography, stamping techniques, embossing techniques, molding techniques, microetching techniques, etc. As will be appreciated by those in the art, the technique used will depend on the composition and shape of the substrate 50A and the interposer 51.

The membrane 56 may be any of the non-permeable or semi-permeable materials described herein. In this example, the membrane 56 is positioned between the second portion 22″ of the cis well 22 and the trans wells 26, and thus provides a barrier between the wells 22, 26. The membrane 56 may be positioned on regions (e.g., sidewalls 64) of the interposer 51 that define the trans wells 26.

The nanopore(s) 30 may be any of the biological nanopores, solid state nanopores, and hybrid nanopores described herein. As mentioned herein, each nanopore 30 fluidically connects a respective one of the trans wells 26 to the cis well 22. As such, the ratio of nanopores 30 to trans wells 26 is 1:1.

The nanopore 30 has two open ends and a hollow core or hole that connects the two open ends. The walls of the hollow core or hole are an inner surface of the nanopore 30. When inserted into the membrane 56, one of the open ends of the nanopore 30 faces the cis well 22 and the other of the open ends of the nanopore 30 faces the trans well 26 and is aligned with at least a portion of the opening of trans well 26. The hollow core of the nanopore 30 enables the fluidic connection between the wells 22, 26. The diameter of the hollow core may range from about 1 nm up to 1 μm, and may vary along the length of the nanopore 30. In some examples, the open end that faces the cis well 22 may be larger than the open end that faces the trans well 26. In other examples, the open end that faces the cis well 22 may be smaller than the open end that faces the trans well 26.

The nanopore(s) 30 may be inserted into the membrane 56, or the membrane 56 may be formed around the nanopore(s) 30. In an example, the nanopore 30 may insert itself into a formed lipid bilayer (one example of the membrane 56). For example, a nanopore 30 in its monomeric form or polymeric form (e.g., an octamer) may insert itself into the lipid bilayer and assemble into a transmembrane pore. In another example, the nanopore 30 may be added to a grounded side of a lipid bilayer at a desirable concentration where it will insert itself into the lipid bilayer. In still another example, the lipid bilayer may be formed across an aperture in a polytetrafluoroethylene (PTFE) film and positioned between the cis and trans wells 22, 26. The nanopore 30 may be added to the grounded cis compartment, and may insert itself into the lipid bilayer at the area where the PTFE aperture is formed. In yet a further example, the nanopore 30 may be tethered to a solid support (e.g., the trans electrode 28 or the sidewall 57). A tethering molecule, which may be part of the nanopore 30 itself or may be attached to the nanopore 30, may attach the nanopore 30 to the solid support. The attachment via the tethering molecule may be such that a single pore 30 is immobilized in a single trans well 26. A lipid bilayer may then be formed around the nanopore 30.

The nanopore sensor device 10 includes the redox mediator species in the trans well 26 and the redox mediator chamber 18, and includes the buffer solution in the cis well 22. In some examples, the oxidized form of the redox mediator species may be included in the trans well 26 and the reduced form of the redox mediator species may be included in the redox mediator chamber 18 when the trans electrode 26 is the anode (where oxidation takes place) and the cis electrode 20 is the cathode (where reduction takes place). In other examples, the reduced form of the redox mediator species may be included in the trans well 26 and the oxidized form of the redox mediator species may be included in the redox mediator chamber when the trans electrode 26 is the cathode (where reduction takes place) and the cis electrode 20 is the anode (where oxidation takes place). The mechanism/membrane 24 that is selected will enable the desired ion transport between the cis well 22 and the redox mediator chamber 18.

The second portion 22″ of the cis well 22 is capable of maintaining the buffer solution in contact with the nanopore(s) 30, and also enables sequencing biochemistry introduced through the inlet 52 to reach the nanopore(s) 30. The fluid communication through the nanopore(s) 30 is indicated by the arrows in FIG. 1.

FIG. 2A through FIG. 2C depict another example of the nanopore sensing system 10′ that can include the mechanism/membrane 24. This example is similar to the example shown in FIG. 1 except that two substrates 50A, 50B are adhered together to form the system 10′. FIG. 2A illustrates an exploded view of two of the substrates 50A, 50B. For clarity, the interposer 51, the membrane 56, the nanopores 30, and electrodes 20, 28 are not shown in FIG. 2A.

In this example, the portions 22′, 22″ of the cis well 22 are defined in separate substrates 50A, 50B. As shown from the bottom view of the substrate 50B in FIG. 2B, the first portion 22′ of the cis well 22 is defined in the substrate 50B, and as shown from the bottom view of the substrate 50A in FIG. 2C, the second portion 22″ of the cis well 22 is defined in the substrate 50A. In this example, the inlet 52 is defined in the substrate 50B, and the outlet 54 is defined in the substrate 50A. The substrate 50A also includes a flow through passage 68 fluidly connecting the first and second portions 22′, 22″. As such, when the substrate 50A and 50B are adhered together, the portion 22′ of the cis well 22 is positioned such that it overlies the flow through passage 68 so that any buffer solution and sequencing biochemistry introduced into the inlet 52 is able to flow through the passage 68 into the second portion 22″ of the cis well 22. It is to be understood that the inlet 52 and the flow through passage 68 may be offset from one another as shown in FIG. 2A or may be in line with one another (not shown).

While not shown, it is to be understood that in this example, the interposer 51 is a photoresist or other material that can be patterned to form the individual trans wells 26.

FIG. 2A and FIG. 2C also depicts the inlet for the redox mediator chamber 18 (shown at reference numeral 70), the outlet for the redox mediator chamber 18 (shown at reference numeral 72), and the aperture 62. Each of these openings is formed in the substrate 50A. As shown in FIG. 2C, the mechanism/membrane 24 is positioned to cover the aperture 62 from the interior of the redox mediator chamber 18. The mechanism/membrane 24 does not cover the inlet 70 or the outlet 72. When the substrates 50A and 50B are adhered together, the portion 22′ of the cis well 22 is positioned such that i) it overlies the aperture 62 so that ions are able to flow through the aperture 62 into or out of the redox mediator chamber 18, and ii) is does not overlie the inlet 70 or outlet 72 of the redox mediator chamber.

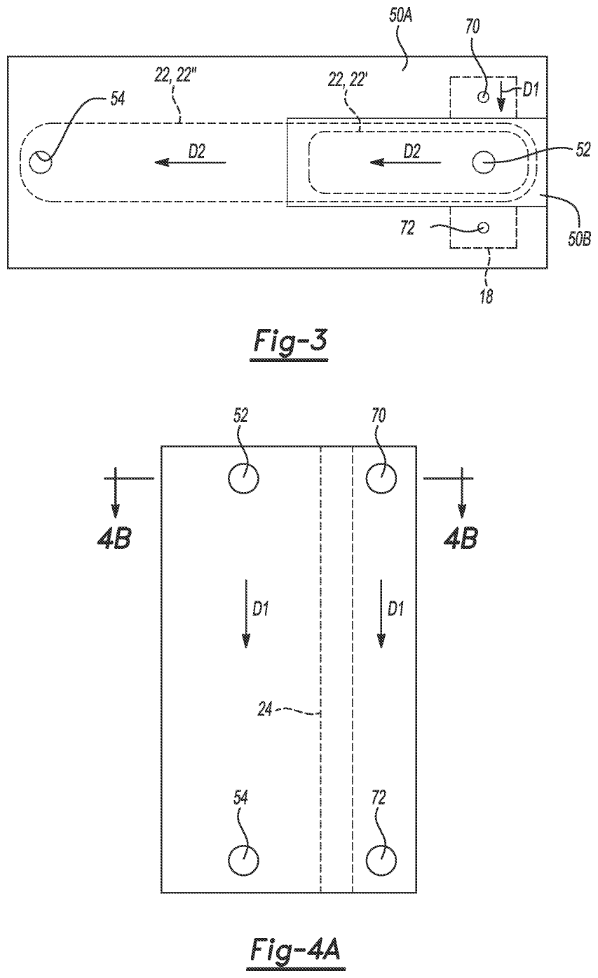

In this example, the redox mediator chamber 18 is positioned so that fluid flow from the inlet 70 to the outlet 72 is in a first direction D1 and the cis well 22 is positioned so that fluid flow from the inlet 52 to the outlet 54 is in a second direction D2 that is perpendicular to the first direction D1. FIG. 3 depicts a top view of the substrates 50A, 50B after they are secured together. This figure illustrates the redox mediator chamber 18 and the portions 22′, 22″ of the cis well 22 in hidden line, showing that the length of the redox mediator chamber 18 and the lengths of each portion 22′, 22″ of the cis chamber 22 are perpendicular to one another, which enables the perpendicular fluid flow directions D1, D2.

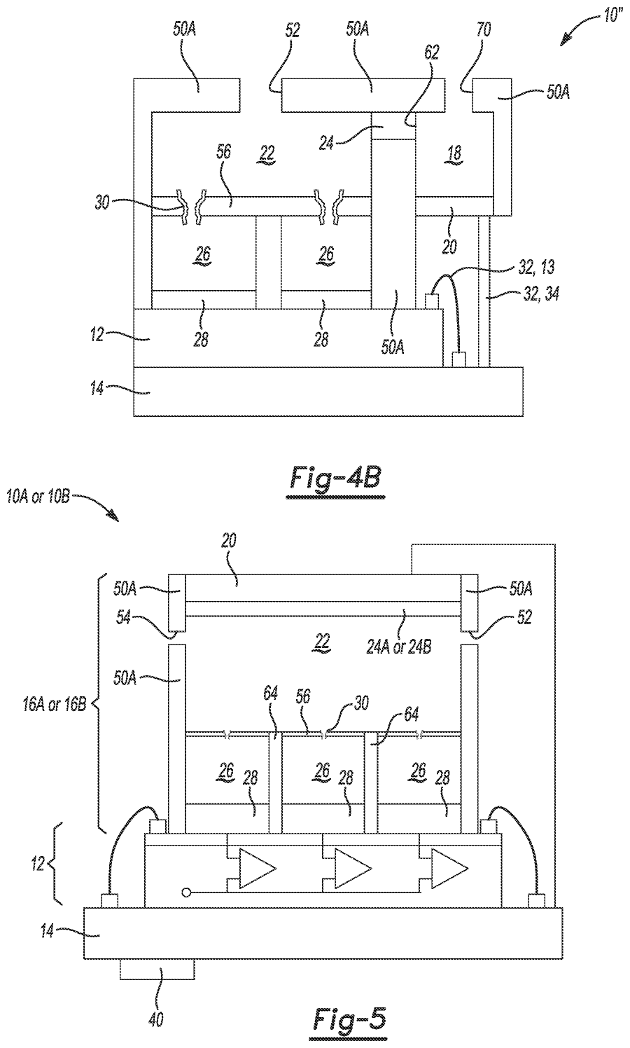

In another example of the nanopore sensing system 10″ (see FIG. 4B), the redox mediator chamber 18 is positioned so that fluid flow from the inlet 70 to the outlet 72 is in a first direction D1 and the cis well 22 is positioned so that fluid flow from the inlet 52 to the outlet 54 is also in the first direction D1. FIG. 4A depicts a top view, and FIG. 4B illustrates a cross-sectional view taken along line 5B-5B of FIG. 4A of a portion of this nanopore sensing system 10″. In this example, the redox mediator chamber 18 and the cis well 22 are parallel to one another, which enables the fluids respectively introduced into the chamber 18 and well 22 to flow in the same direction D1.

In this particular example, the nanopore sensing system 10″ includes a single substrate 50A that defines the redox mediator chamber 18, the cis well 22, the inlets 52, 70, the outlets 54, 72, the trans wells 26, and the aperture 62 between the redox mediator chamber 18 and the cis well 22. While the mechanism/membrane 24 is shown between a portion of the redox mediator chamber 18 and the cis well 22, it is to be understood that the mechanism/membrane 24 could form the entire separator between the chamber 18 and well 22.

The example nanopore sensing systems 10, 10′, 10″ may be formed without the fluids and without the membrane 56 and nanopores(s) 30. In this example, the respective redox mediator species may be introduced into the trans wells 26 and the redox mediator chamber 18, and then the membrane 56 may be formed. The buffer solution may then be introduced into the cis well 22, and the nanopore(s) 30 may be formed.

Referring now to FIG. 5, two additional examples of the nanopore sensing system 10A, 10B are depicted. In these example systems 10A, 10B, a different type of mechanism 24A or 24B is used for the confinement of the redox mediator species.

The system 10A includes the application specific integrated circuit (ASIC) sensor 12 mounted on the printed circuit board 14 having an electrical interface with the ASIC sensor 12; and a nanopore sequencer 16A formed on the ASIC sensor 12, the nanopore sequencer 16A including: a cis well 22; a cis electrode 20 positioned in the cis well 22; a microporous polymer membrane 24A positioned on the cis electrode 20, the microporous polymer membrane 24A to confine a redox mediator species therein and to allow an ionic species to pass between the cis electrode 20 and the cis well 22; a plurality of trans wells 26, each including a trans electrode 28 positioned therein; and a plurality of nanopores 30 respectively fluidically connecting the cis well 22 to each of the plurality of trans wells 26.

The system 10B includes the application specific integrated circuit (ASIC) sensor 12 mounted on the printed circuit board 14 having an electrical interface with the ASIC sensor 12; and a nanopore sequencer 16B formed on the ASIC sensor 12, the nanopore sequencer 16B including: the cis well 22; the cis electrode 20 positioned in the cis well 22; a non-water soluble and redox active polymer 24B positioned on the cis electrode 20, wherein reduction or oxidation of the non-water soluble and redox active polymer 24B drives ion diffusion; a plurality of trans wells 26, each including a trans electrode 28 positioned therein; and a plurality of nanopores 30 respectively fluidically connecting the cis well 22 to each of the plurality of trans wells 26.

In each of the examples shown in FIG. 5, the nanopore sensing system 10A, 10B includes a single substrate 50A that defines a portion of the cis well 22, the inlet 52, the outlets 54, and the trans wells 26 (including sidewalls 64).

Unlike the nanopore sensing systems 10, 10′, 10″, these example systems 10A, 10B include the cis electrode 20 positioned in the cis well 22 as opposed to in a separate redox mediator chamber 18. As shown in FIG. 5, the cis electrode 20 may be physically connected to the substrate 50A so that its interior surface defines an upper surface of the cis well 22. The cis electrode 20 may be physically connected to desirable areas of the substrate 50A, for example, by an adhesive or another suitable fastening mechanism. The interface between the cis electrode 20 and the areas of the substrate 50A may seal the upper portion of the cis well 22.

The example nanopore sensing system 10A includes a microporous polymer membrane 24A as the mechanism for confining the redox mediator species. The microporous polymer membrane 24A may be a polymer of intrinsic microporosity (PIM) that is absorbed to the surface of the cis electrode 20 that faces the cis well 22. One specific example is a microporous polyamine, such as PIM-EA-TB. The microporous polymer membrane 24A is capable of non-covalently binding the redox mediator species, thereby keeping the redox mediator species from leaching out of the membrane 24A.

During fabrication of this example of the nanopore sensing system 10A, the microporous polymer membrane 24A may be applied to the cis electrode 20 before it is secured to the substrate 50A. Once the trans wells 26 are filled with one of the redox mediator species and the coated cis electrode 20 is secured to the substrate 50A, the other redox mediator species (of the redox couple being used) may be introduced into the cis well 22 and allowed to become encapsulated in the polymer membrane 24A. The cis well 22 may be rinsed and flushed before the buffer solution is introduced. Alternatively, the redox mediator species may be entrapped in the microporous polymer before it is coated onto the cis electrode 20. In this example, the microporous polymer and redox mediator species are physically mixed in solution or in the solid phase, allowing the microporous polymer to encapsulate the redox mediator species. Then, the polymer encapsulating with mediator species is coated onto the cis electrode 20 during fabrication.

The example nanopore sensing system 10B includes the non-water soluble and redox active polymer 24B as the redox mediator species and the confinement mechanism. The non-water soluble and redox active polymer 24B participates in the redox reaction as ions are shuttled in and out of the polymer 24B. For the reduction reaction occurring at the cis electrode 20, the reaction could either involve the reduction of a neutral polymer to a negatively charged polymer (P⁰→P⁻) or the reduction of a p-doped polymer to a neutral polymer (P⁺→P⁰) if the cis electrode 20 is the cathode.

In one example, the non-water soluble and redox active polymer 24B has a heterocyclic backbone, such as a poly(thiophene) or poly(pyrrole) backbone that may not be functionalized with a polar side chain (e.g., a glycol). Any of the following monomers may make up the heterocycle backbone:

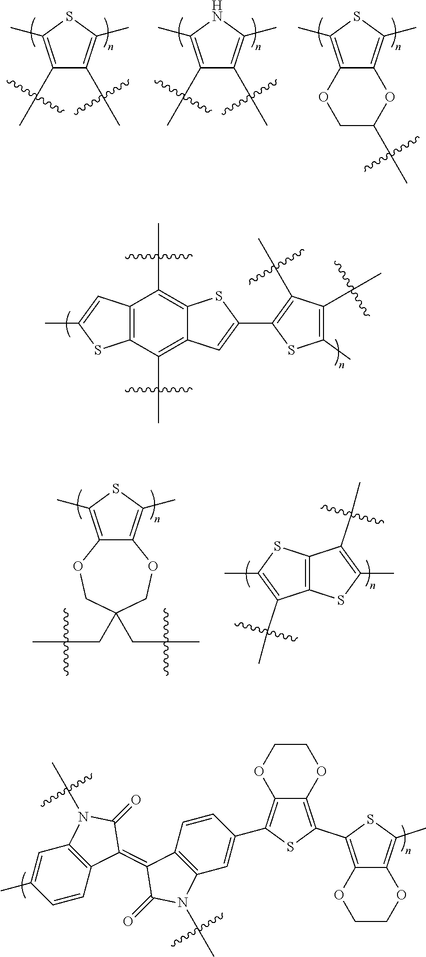

where n ranges from 3 to 1000; and any of the following may make up the polar side chain:

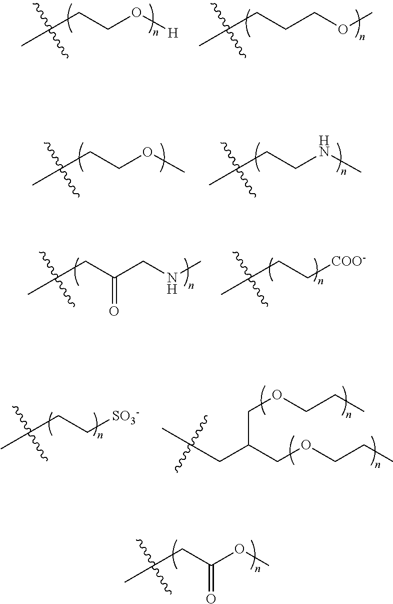

where n can range from 1 to approximately 10. A commercially available example of one such material is poly(3-[2-[2-(2-Methoxyethoxy)ethoxy]ethyl]thiophene-2,5-diyl) from Reike Metals.

It is to be understood that the heterocycle backbone may also be a copolymer of any two or more of the backbone monomers, or may be a derivative of any of the backbone monomers. As examples, suitable thiophene derivatives may include dioxythiophene and alkylated thiophene. Similarly, a suitable pyrrole derivative may include alkylated pyrrole or sulfonated pyrrole.

In another example, the non-water soluble and redox active polymer 24B has a benzimidazole derived backbone, such as poly(benzimidazobenzophenanthroline) shown below:

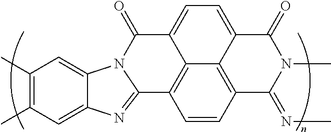

where n ranges from 3 to 1000. This example polymer may include any of the polar side chains set forth herein.

In still another example, the non-water soluble and redox active polymer 24B is poly(3,4-ethylenedioxythiophene) polystyrene sulfonate or polyaniline.

In each of these examples of the non-water soluble and redox active polymer 24B, the polymer backbone is the redox-active component of the polymer. In another example, the non-water soluble and redox active polymer 24B has a redox inert backbone that is functionalized with pendant redox centers (i.e., pendant redox active groups). Such redox centers may be comprised of a radical species, such as (2,2,6,6-Tetramethylpiperidin-1-yl)oxyl (TEMPO), an organic species, such as viologen, pyrene, or anthraquinone, or a metal coordination complex. In the case of the metal coordination complex, the metal atom in the complex uld be a transition metal such as osmium, cobalt, iron, iridium, or copper. The coordination complex may also be metallocene, such as ferrocene or cobaltocene. Suitable non-limiting examples of polymers with pendant redox centers are shown below:

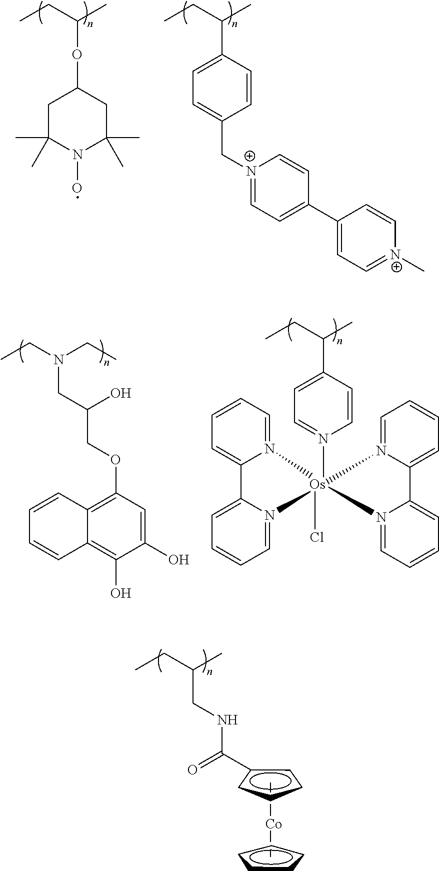

where n ranges from 3 to 1000.

During fabrication of this example of the nanopore sensing system 10B, the non-water soluble and redox active polymer 24B may be electropolymerized onto the cis electrode 20 or cast from solution onto the cis electrode 20.

Referring now to FIG. 12, a block diagram is depicted, illustrating an example of a bioassay system 100 that includes any example of the nanopore sequencing system 10, 10′, 10″, 10A, 10B disclosed herein. The term “bioassay” is not intended to be limiting as the bioassay system 100 may operate to obtain any information or data that relates to at least one of a biological or chemical substance as described herein. In some examples, the bioassay system 100 is a workstation that may be similar to a bench-top device or desktop computer. For example, a majority (or all) of the systems and components for conducting the designated reactions can be within a common housing 116.

In particular examples, the bioassay system 100 is a nucleic acid sequencing system (or sequencer) that can perform various applications, including de novo sequencing, resequencing of whole genomes or target genomic regions, and metagenomics. The bioassay system 100 may also be used for DNA or RNA analysis.

The bioassay system 100 may include a system receptacle or interface 102 that can interact with the nanopore sequencing system 10, 10′, 10A, 10B to perform designated reactions within the nanopore sequencing system 10, 10′, 10″, 10A, 10B. In the following description with respect to FIG. 12, the nanopore sequencing system 10, 10′, 10″, 10A, 10B is loaded into the system receptacle 102 so that the connector 40 electrically connects to the field programmable gate array (FPGA) 42 of the sequencing instrument. The bioassay system 100 may perform a large number of parallel reactions within the nanopore sequencing system 10, 10′, 10″, 10A, 10B.

The bioassay system 100 may include various components, assemblies, and systems (or sub-systems) that interact with each other to perform examples of the method disclosed herein. For example, the bioassay system 100 includes a system controller 104 that may communicate with the various components, assemblies, and sub-systems of the bioassay system 100 and also the nanopore sequencing system 10, 10′, 10″, 10A, 10B.

In some of the examples disclosed herein, the system controller 104 is connected to the circuitry 32 of the nanopore sequencing system 10, 10′, 10″, 10A, 10B.

Other components, assemblies, and sub-systems of the bioassay system 100 may include a fluidic control system 106 to control the flow of fluid throughout a fluid network of the bioassay system 100 and the nanopore sequencing system 10, 10′, 10″, 10A, 10B; and a fluid storage system 108 to hold all fluids (e.g., gas or liquids) that may be used by the bioassay system 100.

The bioassay system 100 may also include a user interface 114 that interacts with a user. For example, the user interface 114 may include a display 113 to display information for or request information from the user, and a user input device 115 to receive user inputs. In some examples, the display 113 and the user input device 115 may be the same device. For example, the user interface 114 may include a touch-sensitive display to detect the presence of an individual's touch and also to identify a location of the touch on the display. However, other user input devices 115 may be used, such as a mouse, touchpad, keyboard, keypad, handheld scanner, voice-recognition system, motion recognition system, and/or the like.

The bioassay system 100 may communicate with various components, including the nanopore sequencing system 10, 10′, 10″, 10A, 10B, to perform the designated reactions. The bioassay system 100 may also be configured to analyze data obtained from the nanopore sequencing system 10, 10′, 10″, 10A, 10B to provide a user with desired information.

The system controller(s) 104 may include any processor-based or microprocessor based system, including systems using microcontrollers, reduced instruction set computers (RISC), application specific integrated circuits (ASICs), field programmable gate array (FPGAs), logic circuits, and any other circuit or processor that can execute functions described herein. While several examples have been provided, it is to be understood that these are not intended to limit in any way the definition and/or meaning of the term system controller. In an example, the system controller 104 executes a set of instructions that are stored in one or more storage elements, memories, or modules in order to operate the nanopore sequencing system 10, 10′, 10″, 10A, 10B. In an example, the system controller(s) 104 executes a set of instructions that are stored in one or more storage elements, memories, or modules in order to at least one of obtain and analyze detection data. Storage elements may be in the form of information sources or physical memory elements within the bioassay system 100.

The set of instructions may include various commands that instruct the bioassay system 100 or nanopore sequencing system 10, 10′, 10″, 10A, 10B to perform specific operations, such as the methods and processes of the various examples described herein. The set of instructions may be in the form of a software program, which may form part of a tangible, non-transitory computer readable medium or media. As used herein, the terms “software” and “firmware” are interchangeable, and refer to any algorithm and/or computer program stored in memory for execution by a computer. Examples of the memory include RAM memory, ROM memory, EPROM memory, EEPROM memory, and non-volatile RAM (NVRAM) memory.

The software may be in various forms, such as system software or application software. Further, the software may be in the form of a collection of separate programs, or a program module within a larger program or a portion of a program module. The software also may include modular programming in the form of object-oriented programming. After obtaining the detection data, the detection data may be automatically processed by the bioassay system 100, processed in response to user inputs, or processed in response to a request made by another processing machine (e.g., a remote request through a communication link).

While not shown in FIG. 12, it is to be understood that the system controller(s) 104 may be connected to the sensor 10, 10′, 10″, 10A, 10B and the other components of the bioassay system 100 via communication links. The system controller(s) 104 may also be communicatively connected to remote, off-site systems or servers. The communication links may be hardwired or wireless. The system controller(s) 104 may receive user inputs or commands, from the user interface 114 and the user input device 115.

The fluidic control system 106 includes a fluid network, and can be employed to direct and to regulate the flow of one or more fluids through the fluid network. The fluid network may be in fluid communication with nanopore sequencing system 10, 10′, 10″, 10A, 10B and the fluid storage system 108. For example, select fluids may be drawn from the fluid storage system 108 and directed to the nanopore sequencing system 10, 10′, 10″, 10A, 10B in a controlled manner, or the fluids may be drawn from the nanopore sequencing system 10, 10′, 10″, 10A, 10B and directed toward, for example, a waste reservoir in the fluid storage system 108. Although not shown, the fluidic control system 106 may include flow sensors that detect a flow rate or pressure of the fluids within the fluid network. The flow sensors may communicate with the system controller(s) 104.

The fluid storage system 108 is in fluid communication with the nanopore sequencing system 10, 10′, 10″, 10A, 10B, and may store various reaction components or reactants that are used to conduct the designated reactions in the nanopore sequencing system 10, 10′, 10″, 10A, 10B. The fluid storage system 108 may also store fluids for washing or cleaning the fluid network and nanopore sequencing system 10, 10′, 10″, 10A, 10B and for diluting the reactants. For example, the fluid storage system 108 may include various reservoirs to store samples, reagents, enzymes, other biomolecules, buffer solutions, aqueous, and non-polar solutions, and the like. Furthermore, the fluid storage system 108 may also include waste reservoirs for receiving waste products from the nanopore sequencing system 10, 10′, 10″, 10A, 10B.

The system receptacle or interface 102 may engage the nanopore sequencing system 10, 10′, 10″, 10A, 10B in at least one of a mechanical, electrical, and fluidic manner. The system receptacle 102 may hold the nanopore sequencing system 10, 10′, 10″, 10A, 10B in a desired orientation to facilitate the flow of fluid through the nanopore sequencing system 10, 10′, 10″, 10A, 10B. The system receptacle 102 may also include electrical contacts that are able to engage the nanopore sequencing system 10, 10′, 10″, 10A, 10B so that the bioassay system 100 may communicate with the nanopore sequencing system 10, 10′, 10″, 10A, 10B and/or provide power to nanopore sequencing system 10, 10′, 10″, 10A, 10B. Furthermore, the system receptacle 102 may include fluidic ports (e.g., nozzles) that are able to engage the nanopore sequencing system 10, 10′, 10″, 10A, 10B. In some examples, the nanopore sequencing system 10, 10′, 10″, 10A, 10B is removably coupled to the system receptacle 102 in a mechanical manner, in an electrical manner, and also in a fluidic manner.

In addition, the bioassay system 100 may communicate remotely with other systems or networks or with other bioassay systems 100. Detection data obtained by the bioassay system(s) 100 may be stored in a remote database.

To further illustrate the present disclosure, examples are given herein. It is to be understood that these examples are provided for illustrative purposes and are not to be construed as limiting the scope of the present disclosure.

### NON-LIMITING WORKING EXAMPLE

### Example 1

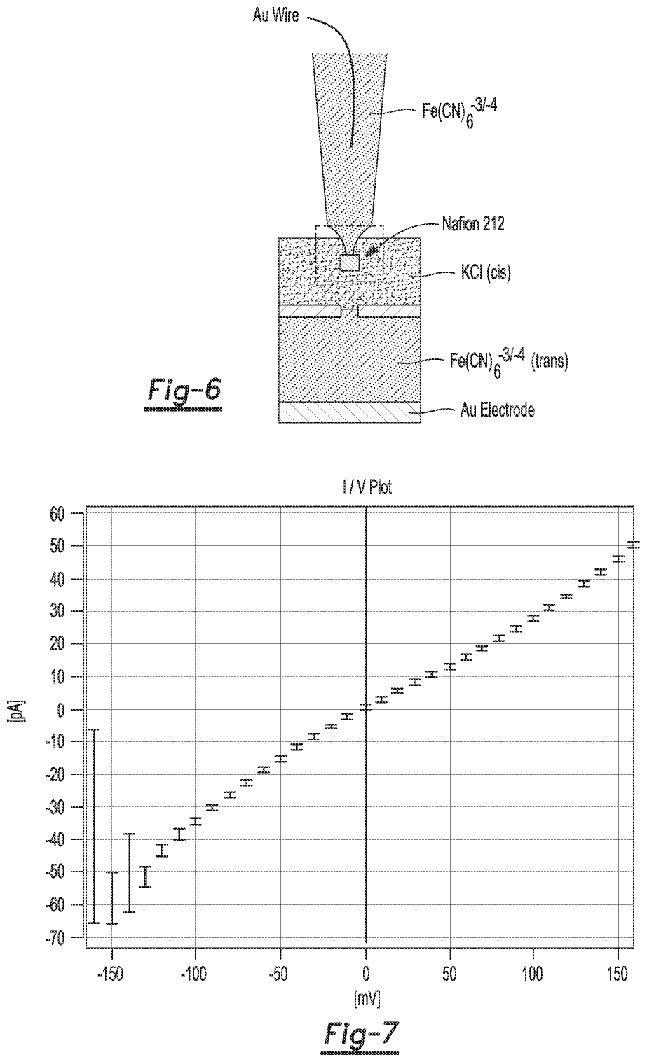

In this example, a NAFION® 212 membrane (thickness of 50.8 μm) was used. NAFION® 212 is based on chemically stabilized perfluorosulfonic acid/PTFE copolymer in acid form, and is available from The Chemours Co. The NAFION® 212 membrane was used to separate the redox mediator species (100 mM of each of potassium ferricyanide and potassium ferrocyanide, and 50 mM HEPES buffer at a pH of 7.5), from the cis buffer solution (150 mM potassium chloride and 50 mM HEPES buffer at a pH of 7.5). The experimental setup (test system) is schematically shown in FIG. 6. In this system, a gold wire cis electrode was positioned in an NMR tube filled with potassium ferri/ferrocyanide. The opening of the NMR tube was covered with the NAFION® 212 membrane, which was sealed with shrink wrap. The covered opening of the NMR tube was placed into a cis well containing potassium chloride, which was separated from a trans well by a lipid bilayer having an MspA pore inserted therein. A gold electrode was positioned in the bottom of the trans well. The trans well was filled with 100 mM of each of potassium ferricyanide and potassium ferrocyanide, and 50 mM HEPES buffer at a pH of 7.5. The cis and trans electrodes were controlled by an automated patch clamp electrophysiology system.

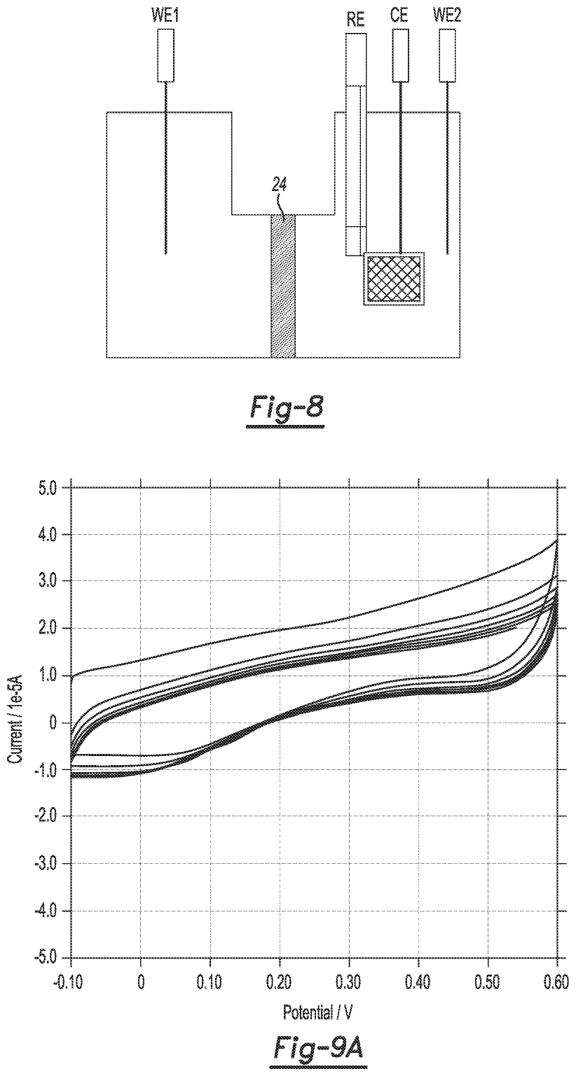

A series of square wave voltage pulses was applied between the cis and trans electrodes. The magnitude of the voltage pulse varied from −150 mV to +150 mV, and the dwell time was 500 ms. As shown in FIG. 7, the average nanopore current at each voltage was detected. The error bars represent the standard deviation of the mean current. At voltages between −120 mV and +150 mV, the current-voltage relationship was substantially linear. At potentials below −120, the current-voltage relationship deviated from linearity, and the standard deviation of the current was substantially increased due to transient ion current blockades. The change in the current-voltage relationship, and the increase in current standard deviation occurring at negative potentials, was consistent with the gating process expected for MspA. This data illustrates the canonical pore behaviour of the nanopore, thus indicating that the pore behaviour is unaffected by NAFION® 212 membrane and that the pore conductance can still be adequately measured in such a system.

### Example 2

In this example, a NAFION™ 117 perfluorosulfonic acid membrane (thickness of 183 μm, from Chemours) was used to separate a solution of the redox mediator species, potassium ferri/ferrocyanide, from the buffer solution, potassium chloride. The experimental setup (test cell) is schematically shown in FIG. 8, where a first well included WE1 (first working electrode used to measure current from ferri/ferrocyanide); and a second well included WE2 (second working electrode used to measure current on the blank side of the NAFION™ 117 membrane), RE (a reference electrode), and CE (a counter electrode). The two working electrodes were controlled by a bipotentiostat. The first well was filled with 1,000 M KCl and 50 mM (4-(2-hydroxyethyl)-1-piperazineethanesulfonic acid) (HEPES buffer) at a pH of 7.5. The second well was filled with 100 mM of each of potassium ferricyanide and potassium ferrocyanide, 400 mM KCl, and 50 mM HEPES buffer at a pH of 7.5.

In the initial test, cyclic voltammetry (CV) was performed using the test cell. The CV sweep was from −100 mV to +600 V at a rate of 50 mV/s. The current at WE1 and at WE2 for the first 10 sweeps (between 2 and 3 minutes) are shown in FIG. 9A and FIG. 9B, respectively. Comparing the data, there was about 1,000 times less current at WE1 than WE2 after the first 10 sweeps.

In the initial test, cyclic voltammetry (CV) was performed using the test cell. The CV sweep for each segment was from −100 mV to +600 V at a rate of 50 mV/s. The current (1 e-3 A, Y axis) versus potential (V, X axi) at WE1 and at WE2 after the first 10 segments (between 2 and 3 minutes) are shown in FIG. 9A and FIG. 9B, respectively. Comparing the data, there was about 1,000 times less current at WE1 than WE2 after the first 10 segments.

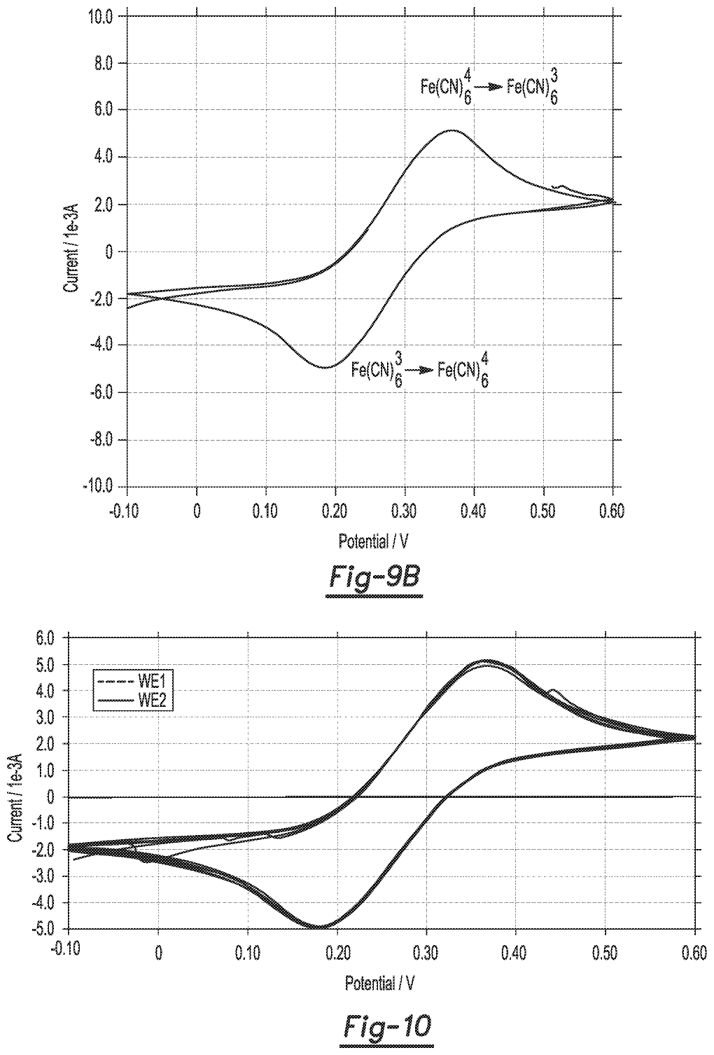

The same CV sweep was continued for 1,000 segments. The current (1 e-3 A, Y axis) versus potential (V, X axis) at WE1 and at WE2 after the first 1,000 segments (about 4 hours) are shown in FIG. 10. Again, there was about 1,000 times less current at WE1 than WE2 after the first 1,000 segments.

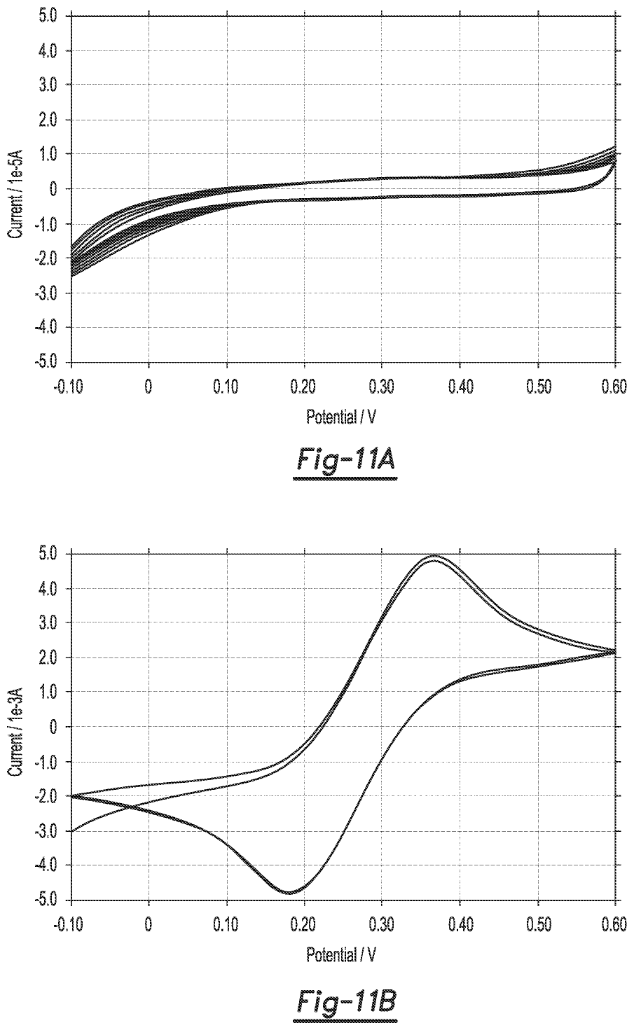

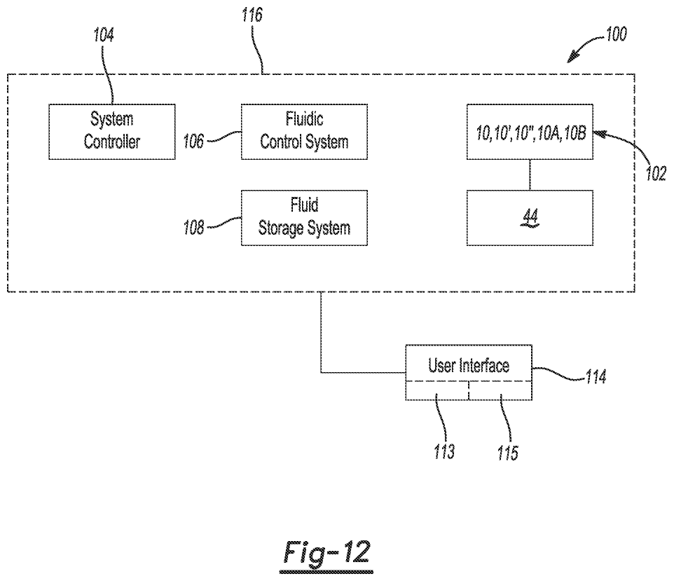

The test cell was then allowed to sit passively for about 12 hours, and then the same CV sweep was performed. The current (1 e-3 A, Y axis) versus potential (V, X axis) at WE1 and at WE2 after 12 hours of passively sitting are shown in FIG. 11A and FIG. 11B, respectively. Comparing the data, there was about 1,000 times less current at WE1 than WE2. The observance of little to no current at WE1 even after 12 hours indicates that little to no ferri/ferrocyanide migrated or diffused across the membrane.

Overall, this example illustrated that the NAFION™ 117 perfluorosulfonic acid membrane stopped the flow of ferri/ferrocyanide when a bias was applied across the membrane.

Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A nanopore sensing system, comprising:
an application specific integrated circuit (ASIC) sensor mounted on a printed circuit board having an electrical interface with the ASIC sensor; and
a nanopore sequencer formed on the ASIC sensor, the nanopore sequencer including:
a redox mediator chamber having a cis electrode positioned therein;
a cis well;
a membrane positioned between the cis well and the redox mediator chamber, the membrane to confine a redox mediator species in the redox mediator chamber and to allow an ionic species to pass between the redox mediator chamber and the cis well;
a plurality of trans wells, each including a trans electrode positioned therein; and
a plurality of nanopores respectively fluidically connecting the cis well to each of the plurality of trans wells.

2. The nanopore sensing system as defined in claim 1, wherein the ASIC sensor is removably mounted on the printed circuit board.

3. The nanopore sensing system as defined in claim 2, wherein the electrical interface includes a pogo pin connection to removably connect the cis electrode to a ground circuit connected to the printed circuit board.

4. The nanopore sensing system as defined in claim 1, wherein the membrane is an ion exchange membrane.

5. The nanopore sensing system as defined in claim 1, wherein the membrane is a size selective membrane.

6. The nanopore sensing system as defined in claim 1, wherein:
the redox mediator chamber includes a first inlet and a first outlet; and
the cis well includes a second inlet and a second outlet.

7. The nanopore sensing system as defined in claim 6, wherein the redox mediator chamber is positioned so that fluid flow from the first inlet to the first outlet is in a first direction and the cis well is positioned so that fluid flow from the second inlet to the second outlet is in a second direction that is perpendicular to the first direction.

8. The nanopore sensing system as defined in claim 6, wherein the redox mediator chamber is positioned so that fluid flow from the first inlet to the first outlet is in a first direction and the cis well is positioned so that fluid flow from the second inlet to the second outlet is also in the first direction.

9. The nanopore sensing system as defined in claim 1, wherein the ASIC sensor is permanently mounted on the printed circuit board.

10. The nanopore sensing system as defined in claim 1, wherein each of the plurality of nanopores is a biological nanopore inserted into a material positioned between the cis well and each of the plurality of trans wells, wherein the material is selected from the group consisting of a material of biological origin and a solid state material.

11. A nanopore sensing system, comprising:
an application specific integrated circuit (ASIC) sensor mounted on a printed circuit board having an electrical interface with the ASIC sensor; and
a nanopore sequencer formed on the ASIC sensor, the nanopore sequencer including:
a cis well;
a cis electrode positioned in the cis well;
a microporous polymer membrane positioned on the cis electrode, the microporous polymer membrane to confine a redox mediator species therein and to allow an ionic species to pass between the cis electrode and the cis well;
a plurality of trans wells, each including a trans electrode positioned therein; and
a plurality of nanopores respectively fluidically connecting the cis well to each of the plurality of trans wells.

12. The nanopore sensing system as defined in claim 11, wherein the ASIC sensor is removably mounted on the printed circuit board.

13. The nanopore sensing system as defined in claim 12 wherein the electrical interface includes a pogo pin connection to removably connect the cis electrode to a ground circuit connected to the printed circuit board

14. The nanopore sensing system as defined in claim 11 wherein the microporous polymer membrane is a microporous polyamine.

15. The nanopore sensing system as defined in claim 11, wherein each of the plurality of nanopores is a biological nanopore inserted into a material positioned between the cis well and each of the plurality of trans wells, wherein the material is selected from the group consisting of a material of biological origin and a solid state material.

16. A nanopore sensing system, comprising:
an application specific integrated circuit (ASIC) sensor mounted on a printed circuit board having an electrical interface with the ASIC sensor; and
a nanopore sequencer formed on the ASIC sensor, the nanopore sequencer including:
a cis well;
a cis electrode positioned in the cis well;
a non-water soluble and redox active polymer positioned on the cis electrode, wherein reduction or oxidation of the non-water soluble and redox active polymer drives ion diffusion;
a plurality of trans wells, each including a trans electrode positioned therein; and
a plurality of nanopores respectively fluidically connecting the cis well to each of the plurality of trans wells.

17. The nanopore sensing system as defined in claim 16, wherein the non-water soluble and redox active polymer has a heterocyclic backbone and a polar side chain.

18. The nanopore sensing system as defined in claim 17, wherein the heterocyclic backbone includes a monomer selected from the group consisting of: 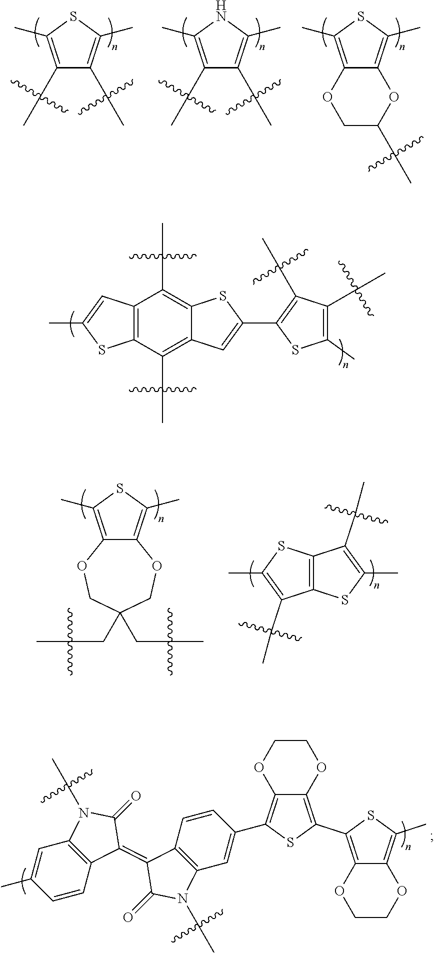
copolymers of two or more of the monomers; or a derivative of any of the monomers.

19. The nanopore sensing system as defined in claim 16, wherein the non-water soluble and redox active polymer has a benzimidazole derived backbone.

20. The nanopore sensing system as defined in claim 16, wherein the non-water soluble and redox active polymer is poly(3,4-ethylenedioxythiophene) polystyrene sulfonate or polyaniline.

21. The nanopore sensing system as defined in claim 16, wherein the non-water soluble and redox active polymer includes a redox inert backbone that is functionalized with pendant redox active groups.

22. The nanopore sensing system as defined in claim 16, wherein the electrical interface includes a pogo pin connection to removably connect the cis electrode to a ground circuit connected to the printed circuit board.

23. The nanopore sensing system as defined in claim 16, wherein each of the plurality of nanopores is a biological nanopore inserted into a material positioned between the cis well and each of the plurality of trans wells, wherein the material is selected from the group consisting of a material of biological origin and a solid state material.
